(19) United States
(12) Patent Application Publication
(10) Pub. No.: US 2005/0080457 A1
(43) Pub. Date: Apr. 14, 2005

(54) Chronically implantable hybrid cannula-electrode system for continuously monitoring electrophysiological signals during infusion of a chemical or pharmaceutical agent

(76) Inventors: Bradley Greger, Van Nuys, CA (US); Babak Kateb, Los Angeles, CA (US)

Correspondence Address: Daniel L. Dawes;MYERS DAWES ANDRAS & SHERMAN LLP, Suite 1150, 19900 MacArthur Boulevard, Irvine, CA, 92612, US

(21) Appl. No.: 10/938,959

(22) Filed: Sep. 10, 2004

Related U.S. Application Data
(60) Provisional application No. 60/505,395, filed on Sep. 23, 2003.

(51) Int. Cl. A61N 1/00; A61K 9/22

(52) U.S. Cl. 607/2; 604/890.1

(57) ABSTRACT

An apparatus for simultaneously measuring electrophysiological signals in a target tissue and for infusing an agent into the target tissue comprises a body, a cannula mounted on the body, and at least one electrophysiological electrode in proximity to the cannula and mounted on the body so that the agent supplied to the cannula is provided to the proximity of the target tissue with which the at least one electrophysiological electrode is electrically coupled. The cannula and electrode are arranged and configured with respect to each in a selected configuration to allow the apparatus to be customized for optimal implantation in specific neurological sites.

### RELATED APPLICATIONS

The present application is related to U.S. Provisional Patent Application, Ser. No. 60/505,395 filed on Sep. 23, 2003, which is incorporated herein by reference and to which priority is claimed pursuant to 35 USC 119.

### BACKGROUND OF THE INVENTION

1. Field of the Invention

The invention relates to the field of electrophysiological implants.

2. Description of the Prior Art

A variety of approaches are required to assess the neuronal mechanisms underlying behavior. Some approaches, such as localized lesions and electrical stimulation, have been used for decades to yield general information about the functions of specific brain structures or pathways. For many years, however, techniques capable of providing information about specific populations of neurons were difficult to apply to behaving animals for most investigations of the mammalian central nervous system. In fact, most recordings of single-unit activity in the rat brain, for example, typically are carried out while the animal is anesthetized and secured in a stereotaxic device.

Similar procedures have been used with in vivo voltammetry, a method designed to obtain neurochemical information on a subsecond time scale. Although the use of these techniques with the stereotaxic preparation continues to provide new insights into brain function, the need to relate electrophysiological and voltammetric data to behavioral events has prompted many laboratories to adapt these recording procedures to freely moving rats.

The key element for successful single-unit and voltammetric recordings from awake, behaving animals is a lightweight, head-mounted, moveable microelectrode assembly. Several such devices have been developed over the years. Although these devices are used widely, they have serious limitations. In some cases, the electrode rotates as it is lowered into brain tissue. This feature not only threatens the integrity of the electrode but also the brain itself as even a slight displacement of the electrode will cause an increasing amount of tissue damage with each rotation. In other cases, the micro-electrode assembly is designed to accommodate metal or glass electrodes, but is not flexible enough to allow either type to be used. Moreover, none of the current designs can be used for voltammetry, which has unique electrode requirements.

In addition, present designs typically do not permit direct pharmacological manipulation of the recording area. Drugs or other chemicals must be administered systemically rather than infused directly into the recording site. Finally, the expense and expertise required to make currently available micromanipulators put them out of reach for laboratories operating on a tight budget.

The prior art has also developed devices for us in neuroscience laboratories with either electrophysiology or voltammetry which permits simultaneous infusions directly into the recording area.

### BRIEF DESCRIPTION OF THE INVENTION

In the illustrated embodiment the invention is primary used for screening of novel pharmacological agents for neural effects or efficacy rather than as a direct medical intervention. The invention is also used in basic neuroscientific research. The device is used to test drugs for any neurologically based pathology, e.g. psychosis (schizophrenia), seizure disorders, sleep/arousal disorders. While this device appears to be primarily directed at pathologies of neuro-electrical activity, it may also be useful in testing drugs for diseases such as Parkinson's and Alzheimer's which may have indirect influences on electrical activity. Also, in every case of the aforementioned diseases, except for Parkinson's, the etiology is unknown. This device is a valuable scientific tool for understanding the mechanisms of neural pathologies.

An apparatus for simultaneously measuring electrophysiological signals in a target tissue and for infusing an agent into the target tissue comprising a body, a cannula mounted on the body, and at least one electrophysiological electrode in proximity to the cannula and mounted on the body so that the agent supplied to the cannula is provided to the proximity of the target tissue with which the at least one electrophysiological electrode is electrically coupled. The cannula and electrode are arranged and configured with respect to each in a selected configuration to allow the apparatus to be customized for optimal implantation in specific neurological sites.

The electrophysiological electrode is biocompatible and adapted for chronic or acute use. The apparatus further comprises a plurality of such electrophysiological electrodes, which are arranged and configured on the body into predetermined array. The illustrated embodiment shows a linear array of electrophysiological electrodes.

The apparatus further comprises an electrode plate coupled to the body for mounting and positioning the electrophysiological electrode(s) into a predetermined array.

The body comprises a manifold for communicating fluid from an external source of the agent to the cannula. A side port is defined in the manifold for providing fluidic communication to the external source.

The apparatus further comprises an electrical connector coupled to the electrophysiological electrode(s).

The invention is also a method performed by using the above apparatus. The method comprises the steps of coupling with an electrophysiological signal with at least one electrophysiological electrode in a target tissue, and simultaneously infusing an agent into the target tissue though a cannula provided in proximity of the target tissue with which the at least one electrophysiological electrode is electrically coupled.

The method further comprises coupling with a plurality of electrophysiological signals with a corresponding plurality of electrophysiological electrodes in the target tissue, namely sensing the electrophysiological signals from the target tissue, or inserting the electrophysiological signals into the target tissue.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side elevational view of the implant of the invention.

FIG. 2 is a diagrammatic view of the implant showing the electrical connection of the electrodes to the interface.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The invention is a chronically implantable hybrid cannula-electrode system 10 for the continuous monitoring of electrophysiological signals during the infusion of chemical and/or pharmacological agents. This system 10 is useful in testing the short-term and long-term effects of drug on electrically active tissues, e.g. the effects of anti-depressants on neuronal activity in the cerebral cortex.

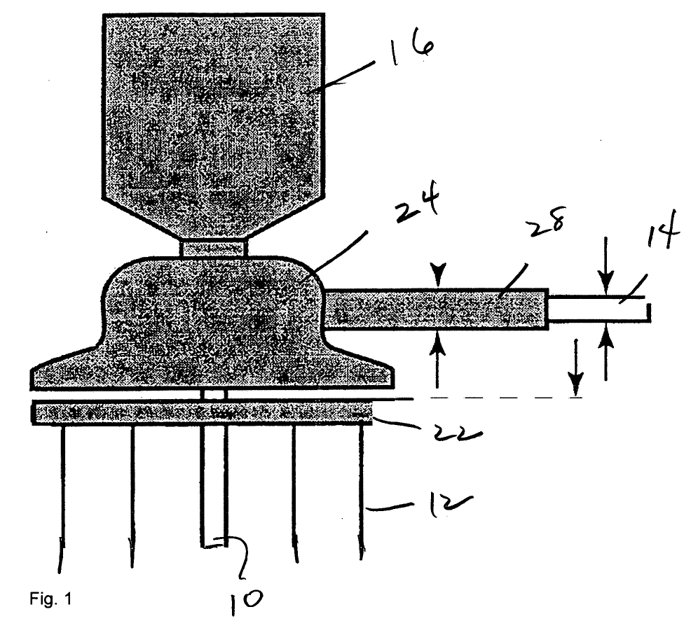

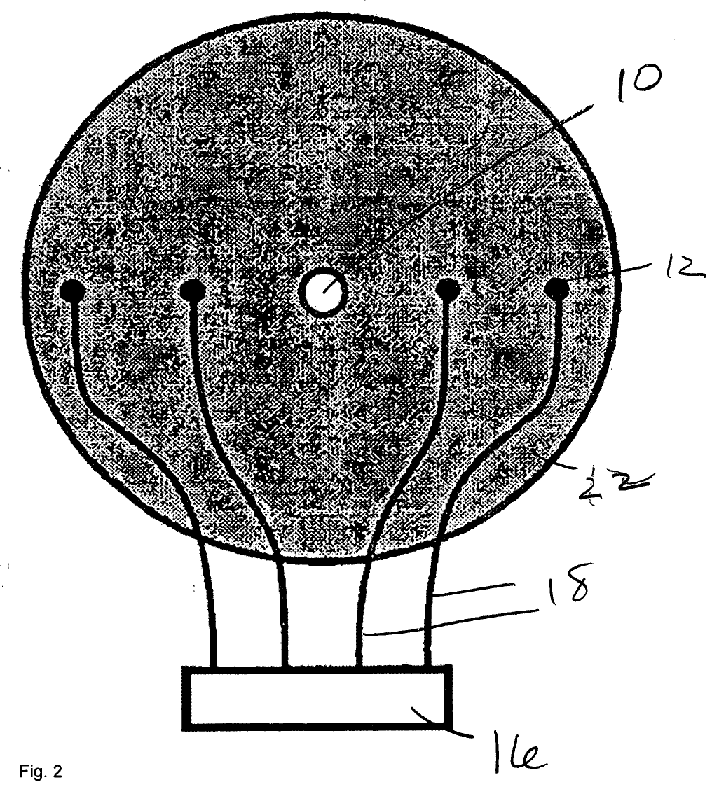

FIG. 1 is a side elevational view of implant system 10 showing a manifold 24 with which a hollow cannula 20 and a side port 28 are communicated. Catheter tubing 14 is coupled to side port 28 so that fluid from an external source can be supplied through side port 28 to manifold 24 and thence to cannula 20. As diagrammatically shown in FIGS. 1 and 2 a cannula 20 is flanked by or associated with a plurality of electrodes 12, which are positioned by insulative electrode plate 22. The cannula 10 is connected by means of catheter tubing 14 to an infusion device (not shown) such as an osmotic pump, for the delivery of the chemical and/or pharmacological agents.

Electrode plate 22 is positioned beneath manifold 20 and provides the mechanical mounting for the array of electrodes 12. The illustrated embodiment depicts four electrodes 12, but the number is arbitrary. Further, electrodes 12 can be arranged in a plurality of geometric configurations and all of which are within the scope of the invention. FIGS. 1 and 2 illustrate a linear array of electrodes 12 by way of example. The electrodes 12 are wired through wires 18 to an electrical interface 16 diagrammatically depicted in FIG. 2 and illustrated in side elevational view in FIG. 1 to allow connection to amplifiers, filters, and data acquisition hardware for the recording of electrophysiological signals. Any type of multiple contact electrical connector or telemetry circuit now known or later devised can be provided on interface 16.

In the linear array of FIGS. 1 and 2 cannula 20 is approximately 2.0 mm long and electrodes 12 are approximately 2.5 mm long. The diameter of electrode plate 22 and manifold 24 is approximately 5.9 mm and the overall height of the device or system 10 from the lower end of electrodes 12 to the upper end of interface 16 is approximately 8.8 mm. Clearly other dimensions could have been chosen without departing from the spirit and scope of the invention.

We have successfully implanted units into the frontal and parietal cortexes of rats. Both electrophysiological and histological data was obtained from these animals. The device has proven itself in the acquisition of data in rats. We have both electrophysiological and histological data from several rats used to study the effects of anti-inflammatory drugs on the long-term quality of electrical recordings. The device 10 is surgically implanted through a small opening in the skull, either by making a small burr hole or by craniectomy. The duramater also will be microsurgically incised prior to implantation or pierced by the cannula(i) 20 and electrode(s) 12, but is otherwise left intact. The device 10 is anchored to the skull using two titanium screws and small island of surgical acrylic (head-cap). The osmotic pump, which is attached to the apparatus will also be implanted subcutaneously, while the electrical connector or interface 16 is imbedded in the head-cap. The scalp is sutured closed around the head-cap, leaving the electrical connector 16 exposed. Alternatively, it is possible using wireless telemetry to couple to electrodes 12 and to have the entire device 10 installed subcutaneously. A completely subcutaneous installation is advantageous in reducing the risk of infection and discomfort to the animal.

System 10 can also be implanted subcutaneously or surgically implanted into deeper anatomical tissues. System 10 may also be miniaturized and modified using conventional design principles in a manner consistent with the teachings of the invention so that it can be endoscopically implanted into a body. In any case system 10 is usually implanted to allow external access to interface 16 and side port 28.

The free and arbitrary design choices of the cannula and electrode number, length and configuration allows the invention to be configured specifically for a targeted biological structure. In the illustrated embodiment, the microelectrodes 12 were manufactured from highly biocompatible materials such as platinum, iridium, or Paralene-C. However, electrode materials and construction could also be arbitrarily chosen according to the teachings and scope of the invention for different biological structures.

It should be noted that the invention contemplates within its scope the use and implantation of multiple infusion pumps, each with different rates of infusion and/or different agents. In such an embodiment different sets of electrodes are associated and operated with operation of the different pumps.

It can now be appreciated that one of the advantages of the invention is the flexibility of its construction. The electrode(s) 12 and cannula(i) 20 can be arrange in virtually any configuration, which allows the device 10 to be easily customized for implantation in specific brain areas. Additionally, the design of the invention gives the user the ability to implant the device completely subcutaneously, using telemetry coupled to an external receiver and osmotic pump(s) which are referred to as an external source above. In the case of subcutaneous implantation the source of fluid or agent is external to the device 10, but internal to the animal, i.e. a reservoir (not shown) holding or storing the agent is also implanted. It is also possible the agent or fluid source could also be external to the animal. In sum, the device is a highly configurable matrix of electrodes 12 and cannuli 20 which is easy to implant both acutely and chronically.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. An apparatus for simultaneously measuring electrophysiological signals in a target tissue and for infusing an agent into the target tissue comprising:
a body;
a cannula mounted on the body; and
at least one electrophysiological electrode in proximity to the cannula and mounted on the body so that the agent supplied to the cannula is provided to the proximity of the target tissue with which the at least one electrophysiological electrode is electrically coupled, the cannula and electrode being arranged and configured with respect to each in a selected configuration, which allows the apparatus to be customized for optimal implantation in specific neurological sites.

2. The apparatus of claim 1 where the electrophysiological electrode is biocompatible and adapted for chronic or acute use.

3. The apparatus of claim 1 further comprising a plurality of electrophysiological electrodes.

4. The apparatus of claim 3 where each of the electrophysiological electrodes of the plurality of electrophysiological electrodes is biocompatible and adapted for chronic or acute use.

4. The apparatus of claim 3 where the plurality of electrophysiological electrodes are arranged and configured on the body into predetermined array.

5. The apparatus of claim 5 where the predetermined array is a linear array of electrophysiological electrodes.

6. The apparatus of claim 1 further comprising an electrode plate coupled to the body for mounting and positioning the electrophysiological electrode.

7. The apparatus of claim 3 further comprising an electrode plate coupled to the body for mounting and positioning the plurality of electrophysiological electrodes into a predetermined array.

8. The apparatus of claim 1 where the body comprises a manifold for communicating fluid from an external source of the agent to the cannula.

9. The apparatus of claim 9 further comprising a side port defined in the manifold for providing fluidic communication to the external source.

10. The apparatus of claim 1 further comprising an electrical connector coupled to the electrophysiological electrode.

11. The apparatus of claim 3 further comprising an electrical connector coupled to the plurality of electrophysiological electrodes.

12. The apparatus of claim 9 further comprising an electrical connector mounted on the manifold and coupled to the electrophysiological electrode.

13. The apparatus of claim 9 further comprising a plurality of electrophysiological electrodes and further comprising an electrical connector mounted on the manifold and coupled to the electrophysiological electrodes.

14. A method comprising:
coupling with an electrophysiological signal with at least one electrophysiological electrode in a target tissue; and
simultaneously infusing an agent into the target tissue though a cannula provided in proximity of the target tissue with which the at least one electrophysiological electrode is electrically coupled.

15. The method of claim 15 further comprising coupling with a plurality of electrophysiological signals with a corresponding plurality of electrophysiological electrodes in the target tissue.

16. The method of claim 16 where coupling with a plurality of electrophysiological signals comprises sensing the electrophysiological signals from the target tissue.

17. The method of claim 16 where coupling with a plurality of electrophysiological signals comprises inserting the electrophysiological signals into the target tissue.

18. The method of claim 17 where sensing the electrophysiological signals from the target tissue comprises sensing the electrophysiological signals in a predetermined array in the target tissue.

19. The method of claim 17 where sensing the electrophysiological signals from the target tissue comprises sensing the electrophysiological signals from the target tissue over a chronic or acute time period.

20. The method of claim 14 further comprising subcutaneously implanting the device into a subject and telemetering the electrophysiological signal from the target tissue to an external receiver.
